(19) United States
(12) Patent Application Publication
(10) Pub. No.: US 2004/0163987 A1
(43) Pub. Date: Aug. 26, 2004

(54) Automatically opening medical device package and method of manufacture

(76) Inventor: John Allen, Mendota Heights, MN (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/374,440

(22) Filed: Feb. 25, 2003

(51) Int. Cl. B65D 83/10

(52) U.S. Cl. 206/438

(57) ABSTRACT

An automatically opening medical device package that includes upper and lower flexible sheet portions, each having distal and proximal ends and first and second peripheral edges. These flexible sheet portions are adapted to be detachably sealed together along their peripheral edges, thereby providing for the enclosure of a medical device. Also included is a collar attached to the distal ends such that a relative movement of the collar and the proximal ends, that decreases a distance therebetween, causes the flexible sheet portions to pull apart, opening the package. A method of manufacturing an automatically opening medical device package containing a medical device includes positioning a medical device on an upper surface of a flexible sheet of material that also has a lower surface, and first and second ends. Upper and lower collar precursors are attached to the lower surfaces of the first and second ends, respectively. The flexible sheet of material is subsequently folded end-over-end about the medical device, thereby forming upper and lower flexible sheet portions (each with distal and proximal ends) that enclose the medical device. The flexible sheet portions are then detachably sealed together. The collar precursors are rolled-back across the flexible sheet portions, operatively aligned, and sealed together to form a collar that is attached to the distal ends such that a relative movement of the collar and the proximal ends, that decreases a distance therebetween, results in a pulling apart of the flexible sheet portions, thereby exposing at least a portion of the medical device.

### BACKGROUND OF INVENTION

1. Field of the Invention

The present invention relates, in general, to medical device packages and, in particular, to automatically opening medical device packages and methods for manufacturing the same.

2. Description of the Related Art

A variety of medical devices require packaging to, for example, protect the medical device from damage prior to use and to maintain sterility of the medical device. For medical devices that include a skin-piercing element (e.g., a lancet or micro-needle), an associated package should provide for deployment of the skin-piercing element during use while also protecting a user from inadvertent contact with the skin-piercing element following use. Furthermore, single-use disposable medical devices call for a medical device package that is inexpensive and disposable. Although it is conceivable that rigid injection molded medical device packages could be designed to provide protection of medical devices enclosed therein, it is likely that their cost and potentially cumbersome manual deployment (i.e., opening) procedures would be less than ideal.

Still needed in the field, therefore, is an inexpensive medical device package that does not require cumbersome manual opening procedures yet still provides for protection of a medical device enclosed therein and/or a sterility barrier. Furthermore, for medical devices that include a skin-piercing element (e.g., a lancet or micro-needle), a need exists for a medical device package that protects the skin-piercing element from damage or contamination prior to use and that also protects a user from accidental contact with the skin-piercing element following use.

### SUMMARY OF INVENTION

Automatically opening medical device packages according to embodiments of the present invention are inexpensive, provide protection and a sterility barrier for medical devices enclosed therein and do not require cumbersome manual opening procedures. Furthermore, embodiments of automatically opening packages according to the present invention are suitable for use with medical devices that include a skin-piercing element (e.g., a lancet or micro-needle) since the packages serve to protect the skin-piercing element from damage or contamination prior to use, as well as to protect a user from accidental contact with the skin-piercing element following use.

An automatically opening medical device package according to an exemplary embodiment of the present invention includes upper and lower flexible sheet portions, each having a distal end, a proximal end, a first peripheral edge and a second peripheral edge. The upper and lower flexible sheet portions are adapted to be detachably sealed together along at least a segment of their first and second peripheral edges, thereby providing for the enclosing of a medical device (e.g., a biosensor medical device, a lancet medical device or an integrated lancet and biosensor medical device) within the upper and lower flexible sheet portions.

The package also includes a collar (e.g., a rigid collar) attached to the distal ends of the upper and lower flexible sheet portions. The collar is attached in such a way that a relative movement of the collar and the proximal ends of the upper and lower flexible sheet portions, that decreases a distance therebetween, results in a pulling apart of the upper and lower flexible sheet portions. This pulling apart automatically opens the package and exposes (e.g., deploys) at least a portion (e.g., a skin-piercing element portion) of the medical device.

Also provided by the present invention is a method of manufacturing an automatically opening medical device package containing a medical device (e.g., an integrated biosensor and lancet medical device). The method includes positioning at least one medical device on a flexible sheet of material. The flexible sheet of material on which the medical device(s) is positioned has upper and lower surfaces, and first and second ends, with the medical device(s) being positioned on the upper surface between the first and second ends.

The method also includes attaching an upper collar precursor to the lower surface of the first end of the flexible sheet of material and a lower collar precursor to the lower surface of the second end of the flexible sheet of material. The flexible sheet of material is subsequently folded end-over-end about the medical device(s), thereby forming an upper flexible sheet portion and a lower flexible sheet portion that enclose the medical device(s). Each of the upper and lower flexible sheet portions thus formed has both a distal end and a proximal end. The upper flexible sheet portion is then detachably sealed to the lower flexible sheet portion.

The upper and lower collar precursors are rolled-back across the upper flexible sheet portion and lower flexible sheet portion, respectively, such that the upper and lower collar precursors are operatively aligned. The upper and lower collar precursors are then joined together to form a collar attached to the distal ends of the upper and lower flexible sheet portions. The collar is attached in such a manner that a relative movement of the collar and the proximal ends of the upper and lower flexible sheet portions, that decreases a distance therebetween, results in a pulling apart of the upper and lower flexible sheet portions, thereby automatically exposing at least a portion of the medical device.

### BRIEF DESCRIPTION OF DRAWINGS

A better understanding of the features and advantages of the present invention will be obtained by reference to the following detailed description that sets forth illustrative embodiments, in which the principles of the invention are utilized, and the accompanying drawings, of which:

FIG. 1A is a perspective top view of an exemplary embodiment of an automatically opening medical device package according to the present invention;

FIG. 1B is a perspective bottom view of the package of FIG. 1A that illustrates (with dashed lines) the location of an integrated biosensor and lancet medical device within the package;

FIG. 2A is a perspective view of a the package of FIGS. 1A and 11B following automatic opening of the package;

FIG. 2B illustrates with dashed lines the location of the integrated biosensor and lancet medical device within the opened package of FIG. 2A;

FIG. 3 is a flow chart illustrating a sequence of steps in a process according to an exemplary embodiment of the present invention;

FIGS. 4A and 4B are perspective and side views, respectively, depicting the result of a step in the manufacturing of the package shown in FIG. 1A with FIG. 4B representing a view along line 4B-4B of FIG. 4A;

FIGS. 5A and 5B are perspective and side views, respectively, depicting a result of another step in the manufacturing of the package shown in FIG. 1A with FIG. 5B representing a view along line 5B-5B of FIG. 5A; and

FIGS. 6A and 6B are perspective and side views, respectively, depicting a result of yet another step in the manufacturing of the package shown in FIG. 1A with FIG. 6B representing a view along line 6B-6B of FIG. 6A.

### DETAILED DESCRIPTION OF THE INVENTION

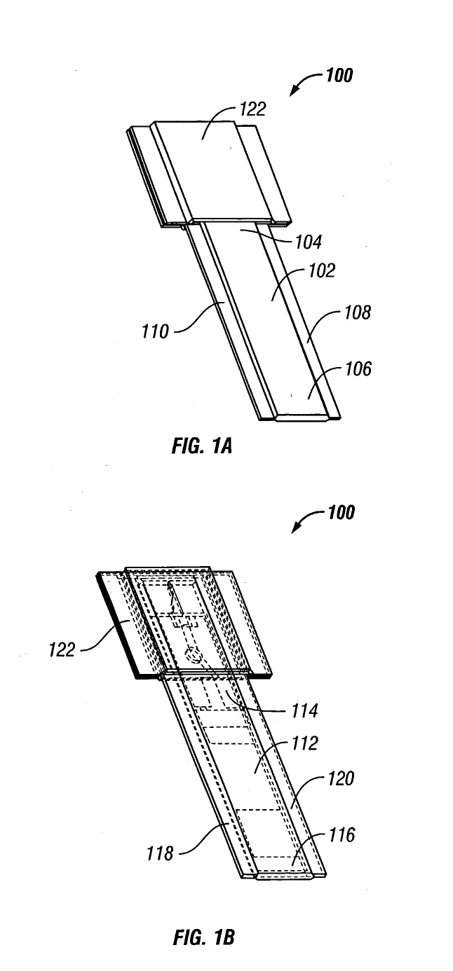

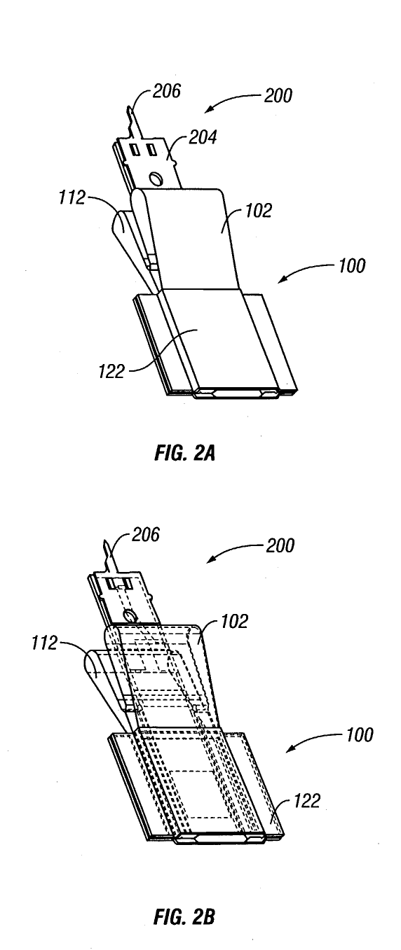

FIG. 1A is a perspective view of an automatically opening medical device package 100 according to an exemplary embodiment of the present invention. FIG. 1B illustrates, with dashed lines, a medical device 200 (i.e., an integrated biosensor 204 and lancet 206 medical device) enclosed within package 100 using a perspective bottom view. FIG. 2A is a perspective view of the package of FIGS. 1A and 1B following automatic opening of the package. FIG. 2B illustrates, with dashed lines, the location of the integrated biosensor and lancet medical device within the opened package of FIG. 2A.

Once apprised of the present disclosure, one skilled in the art will recognize that a variety of medical devices can be beneficially employed with packages according to the present invention. For example, integrated biosensor and lancet medical devices that can be beneficially employed with the current invention are described in U.S. patent application Ser. No. 10/143,399, which is hereby fully incorporated herein by reference. Moreover, those skilled in the art will recognize that packages according to the present invention are not limited to use with integrated biosensor and lancet medical devices. For example, they can also be used with test strip medical devices of the types described in U.S. Pat. Nos. 5,426,032, 5,526,120, 5,708,247, 6,241,862 and 6,284,125. Furthermore, one skilled in the art will appreciate that such test strip medical devices are not limited to the measurement of glucose but can also be used to measure, for example, ketones, glycated albumin, coagulation parameters and cholesterol of a sample.

Package 100 includes an upper flexible sheet portion 102 having a distal end 104, a proximal end 106, a first peripheral edge 108 and a second peripheral edge 110, as shown in FIGS. 1A through 2B. Package 100 also includes a lower flexible sheet portion 112 having a distal end 114, a proximal end 116, a first peripheral edge 118 and a second peripheral edge 120. The distal ends, proximal ends, first peripheral edges and second peripheral edges of the upper and lower flexible sheet portions are essentially aligned with one another (as shown in the top and bottom views of FIGS. 1A and 1B).

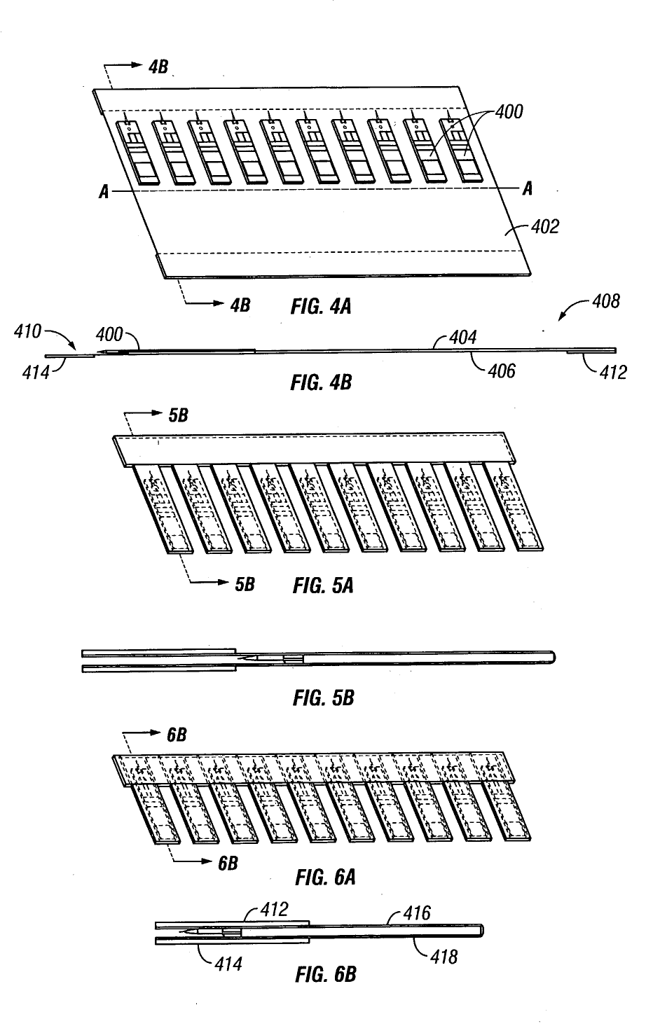

Upper and lower flexible sheet portions 102 and 112 can be formed, for example, from a single sheet of flexible material that has been folded over upon itself (as described further below with respect to FIGS. 4A through 6B). In such a circumstance, proximal ends 106 and 116 are attached to one another along a line where the single sheet of flexible material has been folded.

The upper and lower flexible sheet portions 102 and 112 can be formed from any suitable flexible material known to one skilled in the art. For example, the upper and lower sheet portions can be formed of a flexible paper material (e.g., Kraft Paper, TPP-0036 [HSC 42# Surgical Kraft Paper]), and/or a film-type material (e.g., TPF0504 [Nylon Film—puncture resistant] and 1073B uncoated Tyvek film). Suitable flexible materials are commercially available from, for example, Tolas Health Care Packaging. Furthermore, if desired, the upper and lower flexible sheet portions can be formed of a flexible laminated material such as, for example, a combination of the flexible paper and film-type materials noted above with a metal foil and/or latex coating.

Upper and lower flexible sheet portions 102 and 112 of package 100 are adapted to be detachably sealed together along at least a segment of their first and second peripheral edges. Any suitable adhesive known to one skilled in the art can be employed for this purpose including, but not limited to, cold-seal adhesives, heat-seal adhesives and releasable adhesives available from, for example, 3M, Basic Adhesives and Avery Dennison. In the embodiment of FIGS. 1A, 1B, 2A and 2B, upper and lower flexible sheet portions 102 and 112 of package 100 are also adapted to be detachably sealed together along at least a segment of their proximal and distal ends such that a complete sterility barrier is provided for medical device 200.

In the embodiment of FIGS. 1A through 2B, the sealing of the upper and lower flexible sheet portions provides for enclosing a medical device (e.g., the integrated biosensor and lancet device illustrated in FIGS. 1BA and 2B) within the upper and lower flexible sheet portions. Once the upper and lower flexible sheet portions have been sealed together around the medical device (for example, by sealing the peripheral edges and, optionally, the distal and proximal ends of the upper and lower flexible sheet portions), a sterility barrier can also be provided.

Package 100 also includes a collar 122 attached to the distal ends (104 and 114) of the upper and lower flexible sheet portions (102 and 122, respectively). Collar 122 is attached to the distal ends of the upper and lower flexible sheet portions in such a manner that a relative movement of the collar and the proximal ends of the upper and lower flexible sheet portions, that decreases a distance therebetween, results in a pulling apart of the lower and upper flexible sheet portions. This pulling apart automatically opens the package and exposes (i.e., deploys) at least a portion (e.g., a skin-piercing element portion) of the medical device that had previously been enclosed entirely within the package. In other words, collar 122 is slid toward proximal ends 106 and 116 (or vice versa), thereby pulling apart the upper and lower flexible sheet portions and deploying the enclosed medical device, as illustrated in FIGS. 2A and 2B.

It is envisioned that the relative movement of the collar and the proximal ends can be achieved in a variety of ways. For example, such movement can be induced by a user upon manually moving the collar and/or proximal ends relative to each other. The relative movement can also occur when a user inserts the package into a suitable mechanical interface (e.g., a analytical meter interface), or it can be imposed by a mechanical device (e.g., an analytical meter).

Collar 122 can be formed of any suitable material including, but not limited to, a suitable rigid material (e.g., a rigid plastic film of Mylar and/or polyester) or semi-rigid material. In this regard, a semi-rigid material is a material that has some flexibility but is significantly less flexibility than the upper and lower flexible sheet portions. Although a collar need not be rigid or semi-rigid, a rigid or semi-rigid collar can serve to provide additional protection for an enclosed medical device. In the embodiment of FIGS. 1A through 2B, collar 122 is a rigid collar and therefore adapted to provide impact protection for the lancet 206 of medical device 200 that is encircled by collar 122. Furthermore, the rigid nature of collar 122 serves to facilitate the pulling apart of the upper and lower flexible sheet portions.

In the embodiment of FIGS. 1A through 2B, attachment of collar 122 to the distal ends of the upper and lower flexible sheet portions is such that a subsequent relative movement of the collar and the proximal ends of the upper and lower flexible sheet portions, that increases the distance therebetween, results in an automatic enclosing of the medical device by the upper and lower flexible sheet portions. Such enclosing thereby retracts at least a portion of the medical device, which had been exposed by the prior pulling apart of the upper and lower flexible sheet portions. In other words, once package 100 has been opened (as illustrated in FIGS. 2A and 2B), package 100 can be returned to the configuration shown in FIGS. 1A and 1B by moving collar 122 away from the proximal ends 106 and 116 (or vise versa). Once collar 122 has been returned to the position of FIGS. 1A and 1B, package 100 covers lancet 206 for disposal and protects a user from inadvertent contact with lancet 206.

Automatically opening medical device packages according to the present invention are inexpensive, have a relatively slim profile, and provide a sterility barrier and protection from inadvertent contact with a medical device enclosed therein. Furthermore, humidity protection can also be provided by forming the upper and lower flexible sheet portions from a moisture resistant material (e.g., a metal foil material) and labeling can optionally be applied to the surfaces of the upper and lower flexible sheet portions.

Those skilled in the art will also recognize that automatically opening medical device packages according to the present invention can be secondarily packaged for single use in, for example, a vial or in a cartridge configured for dispensing the automatically opening medical device packages. The secondary package may be constructed of material containing desiccant or may contain separately packaged desiccant for keeping contents moisture free.

Embodiments of the present invention also encompass an automatically opening medical device package and medical device kit that includes automatically opening medical device packages as described above and a medical device (e.g., an integrated biosensor and lancet medical device for measuring blood glucose).

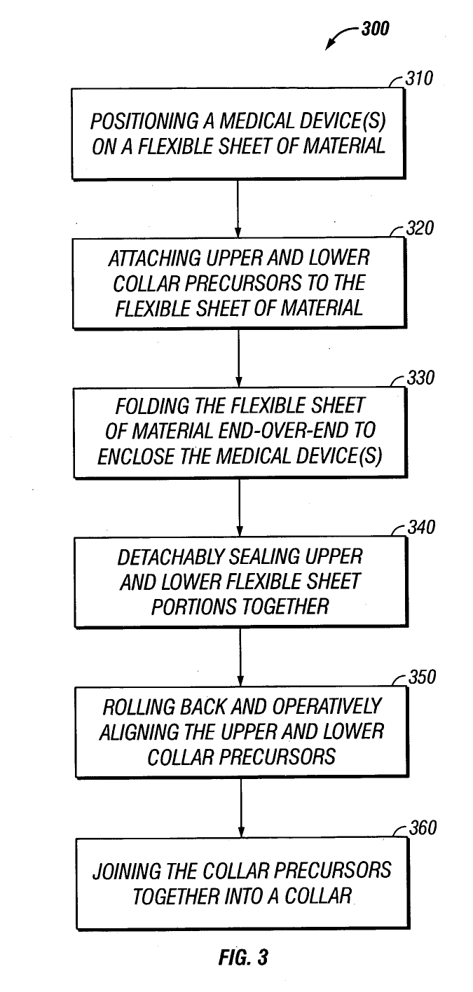

FIG. 3 is a flow chart illustrating a sequence of steps in a process 300 for manufacturing an automatically opening medical device package containing a medical device according to an exemplary embodiment of the present invention. One skilled in the art will recognize that process 300 can be implemented using, for example, web-based manufacturing techniques and other well known conventional manufacturing techniques.

Referring to FIG. 3, FIG. 4A and FIG. 4B, process 300 includes positioning at least one medical device (e.g., an integrated biosensor and lancet medical device 400) on a flexible sheet of material 402, as set forth in step 310 of FIG. 3. Flexible sheet of material 402 can be formed of any suitable material known to one skilled in the art. If desired to provide a sterility barrier for medical device 400, the flexible sheet of material 402 can be formed from a flexible material that is impervious to air and/or air-borne bacteria, such as a thermoplastic flexible material and a metallic foil material.

In the embodiment of FIGS. 4A and 4B, flexible sheet of material 402 has an upper surface 404, a lower surface 406, a first end 408 and a second end 410. In the embodiment of FIGS. 4A through 6B, ten integrated biosensor and lancet devices 400 are positioned on flexible sheet of material 402. In positioning step 310, the medical device(s) is positioned on the upper surface 404 between the first and second ends 408 and 410, respectively.

Next, an upper collar precursor 412 is attached to the lower surface of the first end 408 of the flexible sheet of material and a lower collar precursor 414 is attached to the lower surface of the second end 410 of the flexible sheet of material, as set forth in step 320. The locations of upper and lower collar precursors 412 and 414 are indicated by dashed lines in FIGS. 4A and 5A. Upper and lower collar precursors 412 and 414 can be formed of any suitable material including, for example, rigid materials such as cardboard. The result of steps 310 and 320 of process 300 is depicted in FIG. 4A and FIG. 4B.

Subsequent to the attachment of the upper and lower collar precursors, the flexible sheet of material is folded end-over-end about the medical device(s), thereby forming an upper flexible sheet portion 416 and a lower flexible sheet portion 418 enclosing the medical device(s) 400, as set forth in step 330 of FIG. 3. Folding step 320 can occur, for example, along line A-A of FIG. 4A. Portions of the flexible sheet material between medical devices 400 are then removed. The resultant structure is illustrated in FIGS. 5A and 5B.

Next, the upper flexible sheet portion and lower flexible sheet portion are detachably sealed (using, for example, a heat or pressure sensitive adhesive) to provide protective enclosures for medical devices 400, as set forth in step 340 of FIG. 3. Alternatively, the detachable sealing can occur prior to the removal of portions of the flexible sheet material between medical devices 400.

The upper and lower collar precursors are then rolled-back across the upper flexible sheet portion and lower flexible sheet portion respectively, such that the upper and lower collar precursors 412 and 414 are operatively aligned, as set forth in step 350. The resultant structure is illustrated in FIGS. 6A and 6B.

Next, as set forth in step 360 of FIG. 3, the upper and lower collar precursors are joined together (for example, along outside edges of the upper and lower collar precursors) to form a collar attached to the distal ends of the upper and lower flexible sheet portions in such a way that a relative movement of the collar and the proximal ends of the upper and lower flexible sheet portions, that decreases a distance therebetween, results in a pulling apart of the upper and lower flexible sheet portions. This pulling apart, thereby, automatically exposes and deploys at least a portion of the medical device.

In the circumstance that a plurality of medical devices were positioned on the flexible sheet of material in step 310, each medical device and associated package is singulated by cutting through the collar formed by joining the upper and lower collar precursors. Alternatively, the medical devices and associate packages can be singulated prior to step 360 of FIG. 3.

One skilled in the art will recognize that processes according to the present invention are inexpensive and readily implemented using conventional manufacturing techniques (e.g., web-based manufacturing techniques).

It should be understood that various alternatives to the embodiments of the invention described herein may be employed in practicing the invention. It is intended that the following claims define the scope of the invention and that structures and methods within the scope of these claims and their equivalents be covered thereby.

What is claimed is:

1. An automatically opening medical device package, the package comprising:
an upper flexible sheet portion having a distal end, a proximal end, a first peripheral edge and a second peripheral edge;
a lower flexible sheet portion having a distal end, a proximal end, a first peripheral edge and a second peripheral edge; and
a collar attached to the distal end of the upper flexible sheet portion and the distal end of the lower flexible sheet portion;
wherein the upper and lower flexible sheet portions are adapted to be detachably sealed together along at least a segment of their first and second peripheral edges, thereby providing for the enclosing of a medical device within the upper and lower flexible sheet portions; and
wherein the collar is attached to the distal ends of the upper and lower flexible sheet portions in a manner such that a relative movement of the collar and the proximal ends of the upper and lower flexible sheet portions, that decreases a distance therebetween, results in a pulling apart of the lower and upper flexible sheet portions, thereby automatically opening the package and exposing at least a portion of the medical device.

2. The package of claim 1, wherein the upper and lower flexible sheet portions are adapted to enclose an integrated biosensor and lancet medical device.

3. The package of claim 1, wherein the upper and lower flexible sheet portions are adapted to enclose a biosensor medical device.

4. The package of claim 1, wherein the upper and lower flexible sheet portions are adapted to enclose a lancet medical device.

5. The package of claim 1, wherein the collar encircles a portion of a medical device enclosed within the upper and lower flexible sheet portions.

6. The package of claim 1, wherein the collar is made of a rigid material.

7. The package of claim 6, wherein the collar is made of cardboard.

8. The package of claim 6, wherein the collar is made of a plastic film.

9. The package of claim 1, wherein the collar is made of a semi-rigid material.

10. The package of claim 1, wherein the proximal ends of the upper and lower flexible sheet portions are joined together.

11. The package of claim 1, wherein the upper and lower flexible sheet portions are portions of a single unitary flexible sheet.

12. The package of claim 1, wherein the upper and lower flexible sheet portions are formed of paper.

13. The package of claim 1, wherein the upper and lower flexible sheet portions are formed of a film.

14. The package of claim 1, wherein the upper and lower flexible sheet portions are formed of plastic.

15. The package of claim 1, wherein the upper and lower flexible sheets are detachably sealed using an adhesive coating.

16. The package of claim 1, wherein the collar is attached to the distal ends of the upper and lower flexible sheet portions such that, following the relative movement of the collar and the proximal ends of the upper and lower flexible sheet portions that decreases a distance therebetween, a subsequent relative movement of the collar and proximal ends of the upper and lower flexible sheets, that increases the distance therebetween, results in an automatic enclosing of the medical device by the upper and lower flexible sheet portions.

17. The package of claim 16, wherein the automatic enclosing results in a re-sealing of the upper and lower flexible sheet portions.

18. An automatically opening medical device package and medical device kit, the kit comprising:
a package, the package including:
an upper flexible sheet portion having a distal end, a proximal end, a first peripheral edge and a second peripheral edge;
a lower flexible sheet portion having a distal end, a proximal end, a first peripheral edge and a second peripheral edge; and
a collar attached to the distal end of the upper flexible sheet portion and the distal end of the lower flexible sheet portion;
wherein the upper and lower flexible sheet portions are adapted to be sealed together along at least a segment of their first and second peripheral edges, thereby providing enclosure for a medical device within the upper and lower flexible sheet portions; and
wherein the collar is attached to the distal ends of the upper and lower flexible sheet portions such that a relative movement of the collar and the proximal ends of the upper and lower flexible sheet portions that decreases a distance therebetween results in a pulling apart of the lower and upper flexible sheet portions, thereby exposing any medical device encased within the upper and lower flexible sheet portions; and
a medical device contained within the package.

19. The kit of claim 18, wherein the medical device is an integrated biosensor and lancet medical device.

20. The kit of claim 19, wherein the integrated biosensor and lancet medical device is adapted for the measurement of blood glucose.

21. An automatically opening medical device package and medical device kit, the kit comprising:
a package, the package including:
an upper flexible sheet portion having a distal end, a proximal end, a first peripheral edge and a second peripheral edge;
a lower flexible sheet portion having a distal end, a proximal end, a first peripheral edge and a second peripheral edge; and
a collar attached to the distal end of the upper flexible sheet portion and the distal end of the lower flexible sheet portion;
wherein the upper and lower flexible sheet portions are adapted to be sealed together along at least a segment of their first and second peripheral edges, thereby providing enclosure for a medical device within the upper and lower flexible sheet portions; and
wherein the collar is attached to the distal ends of the upper and lower flexible sheet portions such that a relative movement of the collar and the proximal ends of the upper and lower flexible sheet portions that decreases a distance therebetween results in a pulling apart of the lower and upper flexible sheet portions, thereby exposing any medical device encased within the upper and lower flexible sheet portions, and such that a subsequent relative movement of the collar and proximal ends of the upper and lower flexible sheets, that increases the distance therebetween, results in an automatic enclosing of the medical device by the upper and lower flexible sheet portions; and
a medical device contained within the package.

22. A method for manufacturing an automatically opening medical device package containing a medical device, the method comprising:
positioning at least one medical device on a flexible sheet of material, the flexible sheet of material having an upper surface, a lower surface, a first end and a second end, the at least one medical device being positioned on the upper surface between the first and second ends;
attaching an upper collar precursor to the lower surface of the first end of the flexible sheet of material and a lower collar precursor to the lower surface of the second end of the flexible sheet of material;
folding the flexible sheet of material end-over-end about the at least one medical device, thereby forming an upper flexible sheet portion and a lower flexible sheet portion enclosing the at least one medical device, each of the upper and lower flexible sheet portions having a distal end and a proximal end;
detachably sealing the upper flexible sheet portion to the lower flexible sheet portion;
rolling-back the upper and lower collar precursors across the upper flexible sheet portion and lower flexible sheet portion, respectively, such that the upper and lower collar precursors are operatively aligned; and
joining the upper and lower collar precursors together to form a collar attached to the distal ends of the upper and lower flexible sheet portions in a manner such that a relative movement of the collar and the proximal ends of the upper and lower flexible sheet portions, that decreases a distance therebetween, results in a pulling apart of the upper and lower flexible sheet portions, thereby automatically exposing at least a portion of the medical device.

23. The method of claim 22, wherein the detachably sealing step forms a sterility barrier.

24. The method of claim 22, wherein the positioning step includes positioning a plurality of medical devices.

25. The method of claim 22 further comprising the step of removing portions of the flexible sheet of material after the sealing step and before the rolling-back step.

26. The method of claim 22, wherein the positioning step includes positioning at least one integrated biosensor and lancet medical device.
